(19) United States
(12) Patent Application Publication
(10) Pub. No.: US 2023/0189864 A1
(43) Pub. Date: Jun. 22, 2023

(54) NON-THERAPEUTIC METHODS FOR MAINTAINING A HEALTHY BODY WEIGHT OR LOSING BODY WEIGHT

(71) Applicants: FrieslandCampina Nederland B.V., Amersfoort (NL); Universiteit Maastricht, Maastricht (NL)

(72) Inventors: Ellen Elisabeth BLAAK, Wageningen (NL); Emanuel Enzo CANFORA, Wageningen (NL); Arjen NAUTA, Wageningen (NL)

(21) Appl. No.: 17/614,126

(22) PCT Filed: May 26, 2020
(86) PCT No.: PCT/EP2020/064505

§ 371 Date: Nov. 24, 2021

(30) Foreign Application Priority Data
May 29, 2019 (EP) 19177345.6
Jun. 17, 2019 (EP) 19180567.0

(51) Int. Cl. A23L 33/125; A23L 33/00; A23L 29/212

(52) U.S. Cl. CPC A23L 33/125; A23L 29/212; A23L 33/30; A23L 33/40

(57) ABSTRACT

The invention relates to a non-therapeutic method for maintaining a healthy body weight or losing body weight, in a subject, wherein the non-therapeutic method comprises the step of administering 2′-fucosyllactose and resistant starch, and wherein the subject has a lean weight.

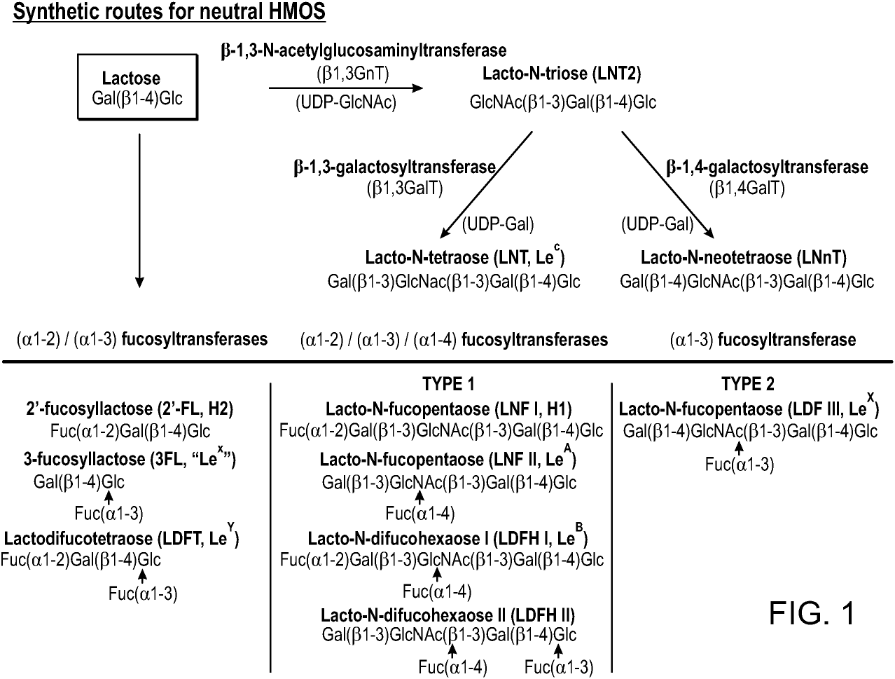

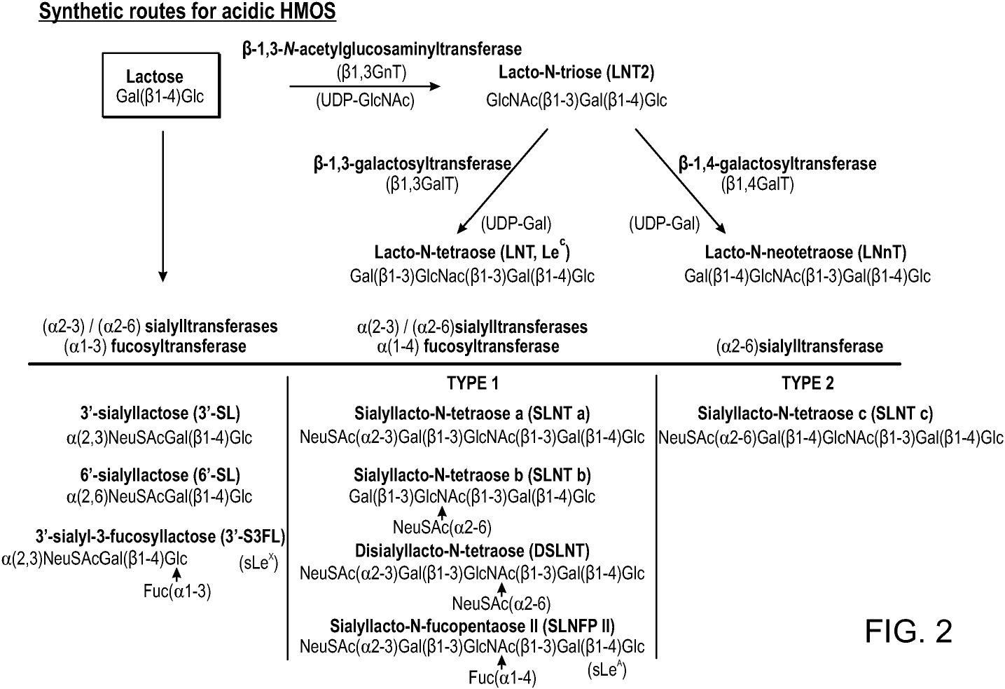

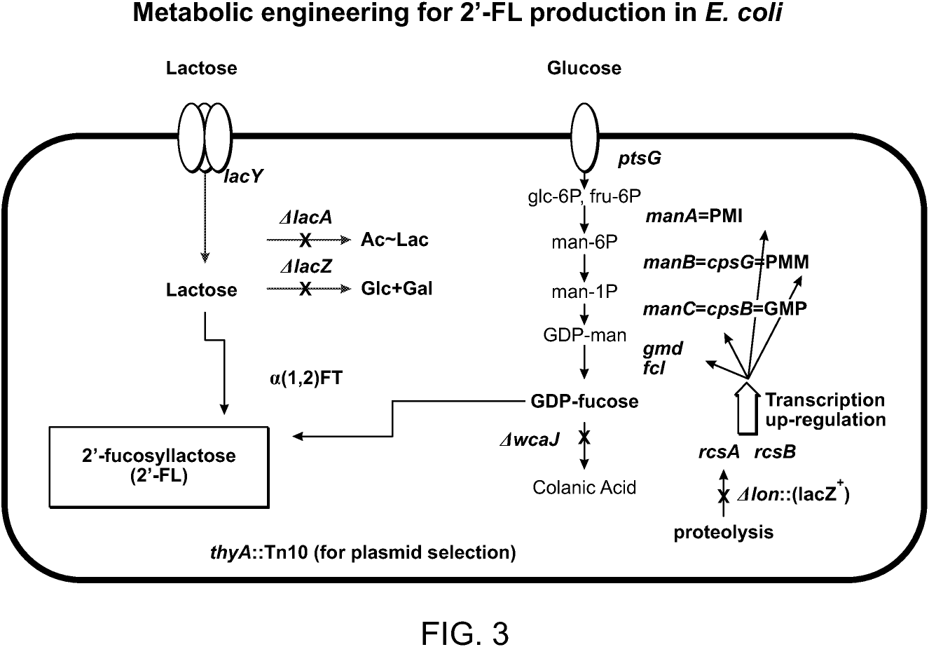

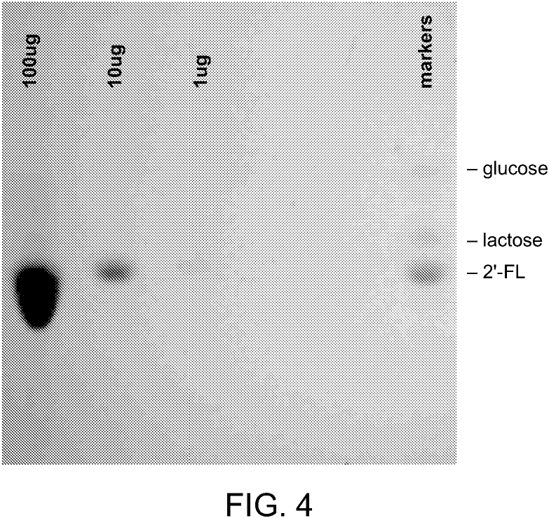

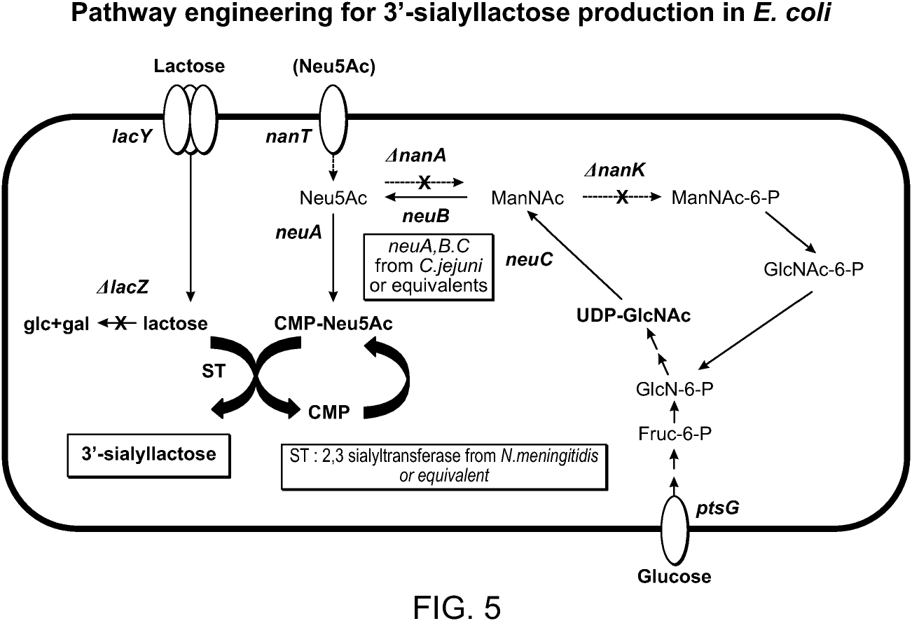

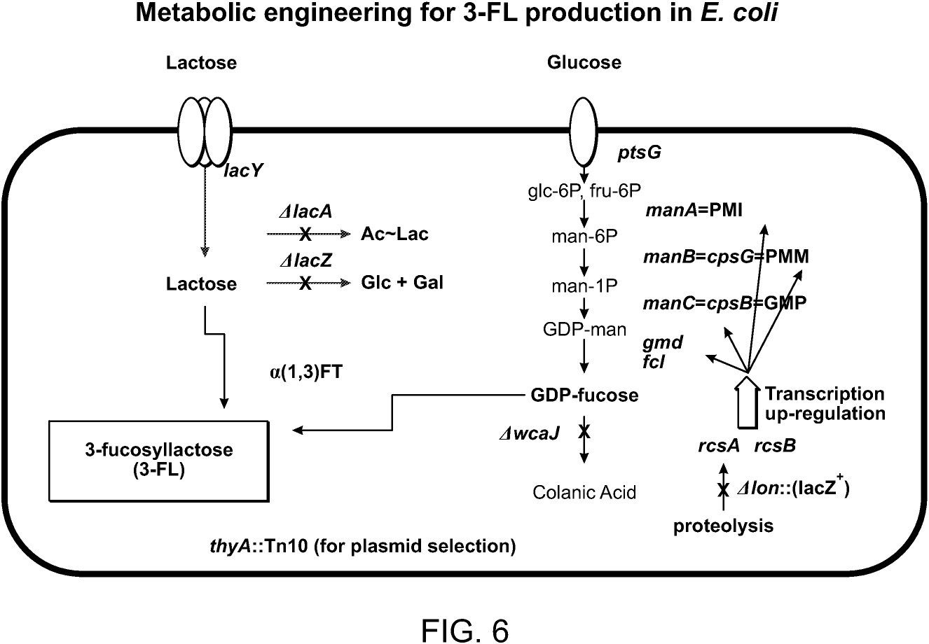

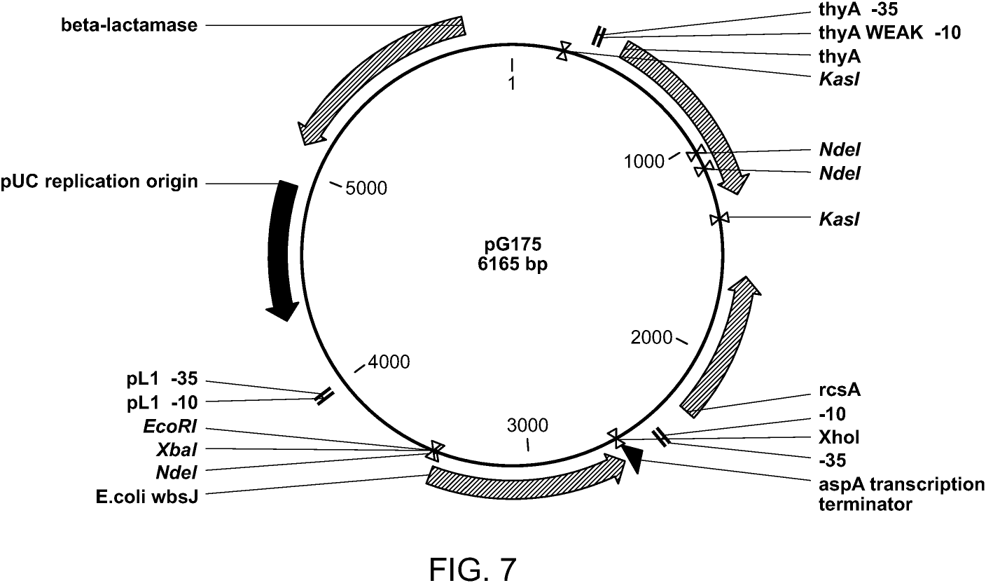

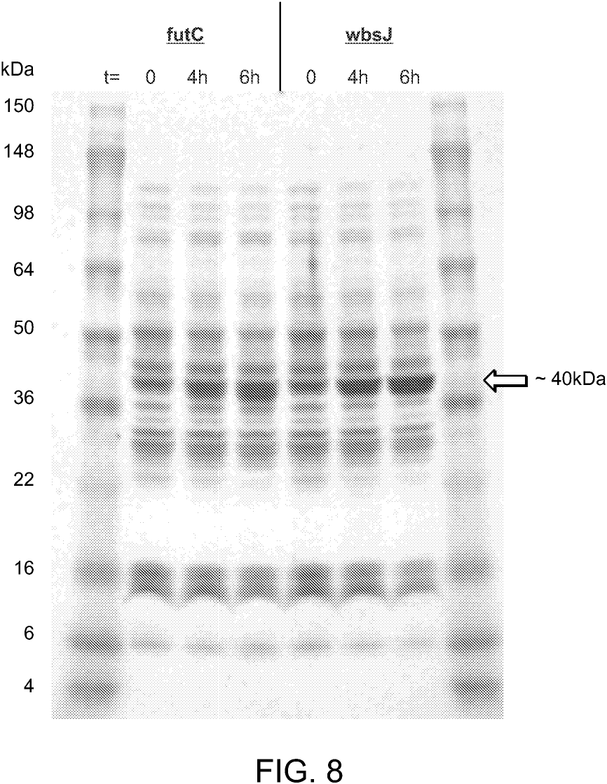

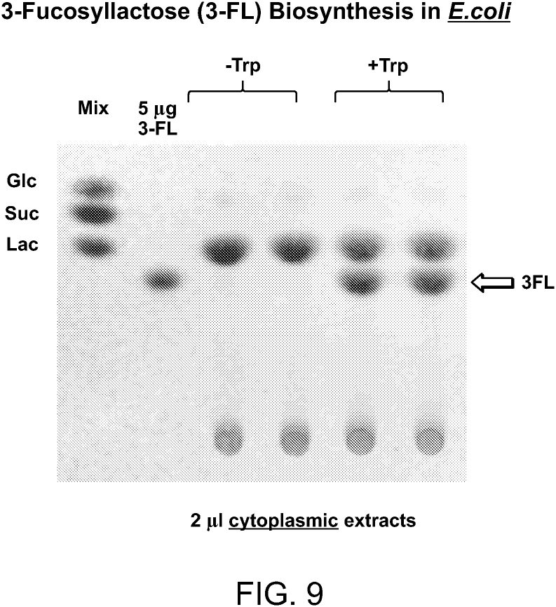

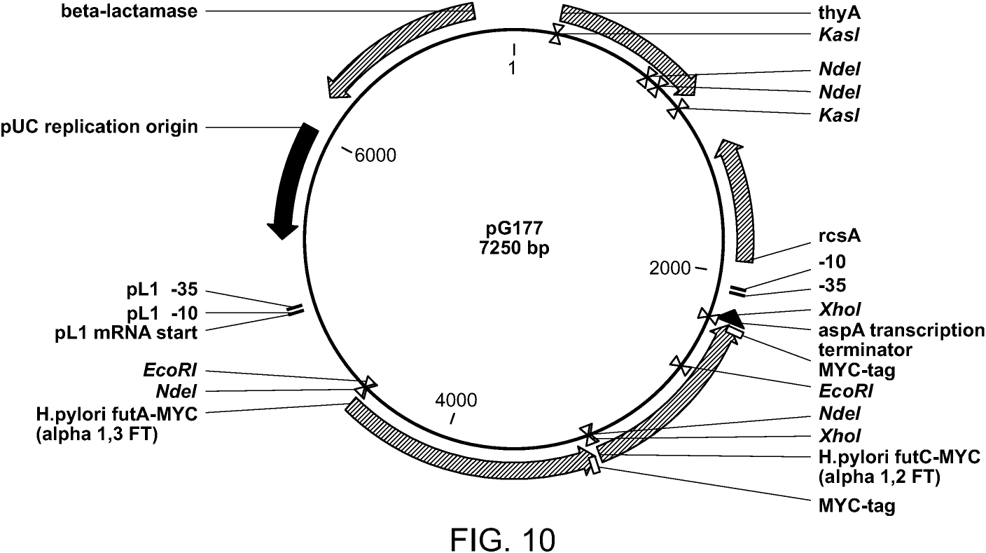

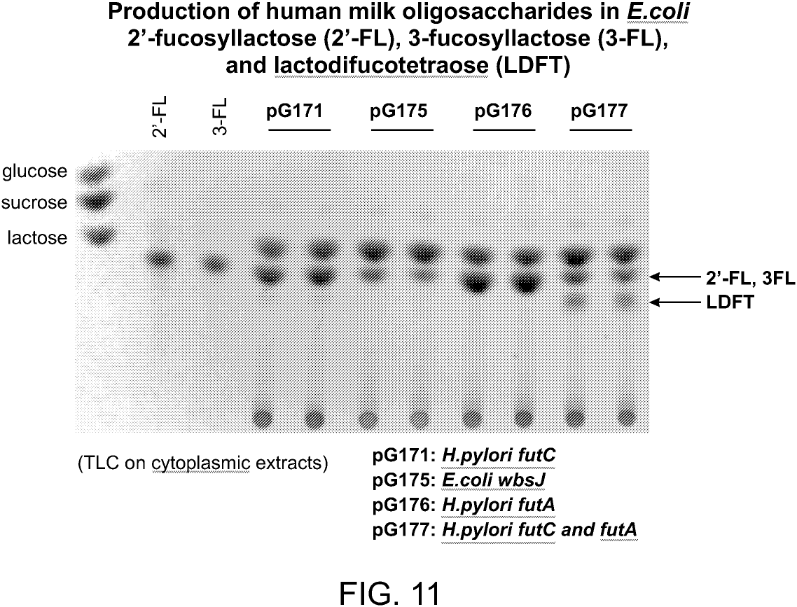

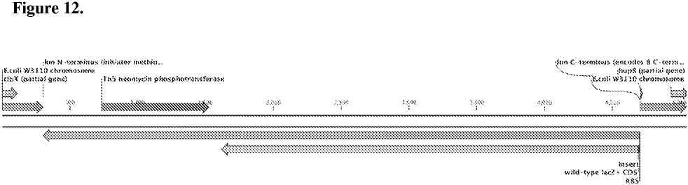

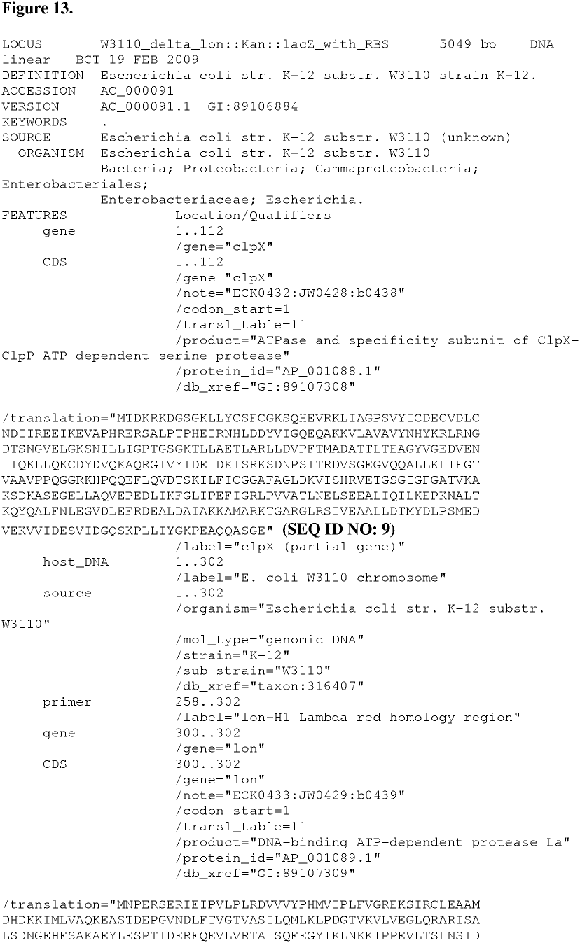

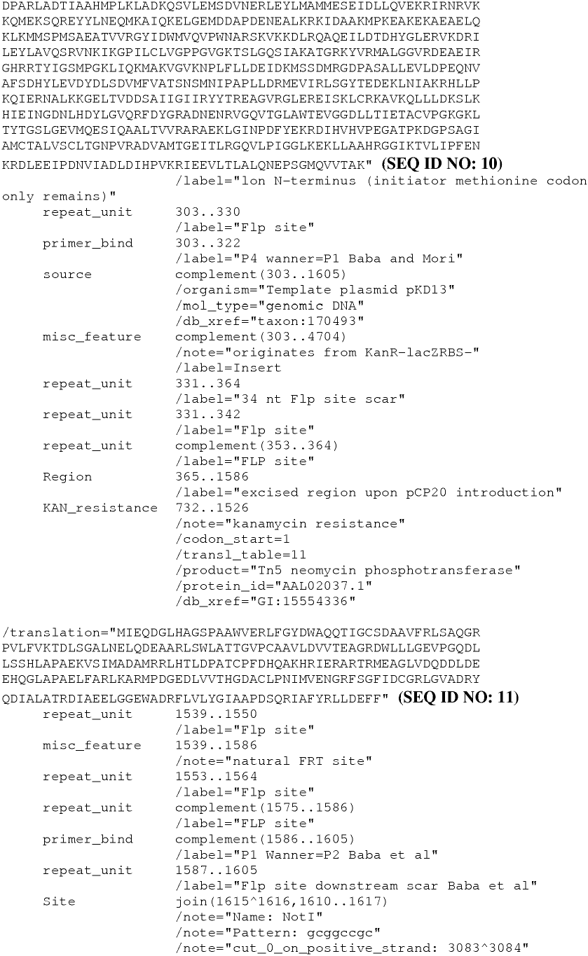

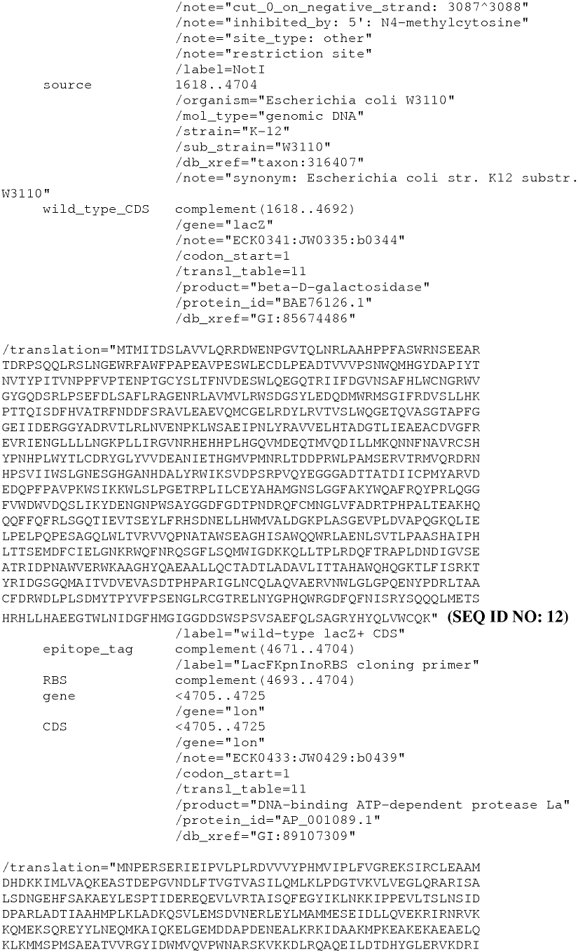

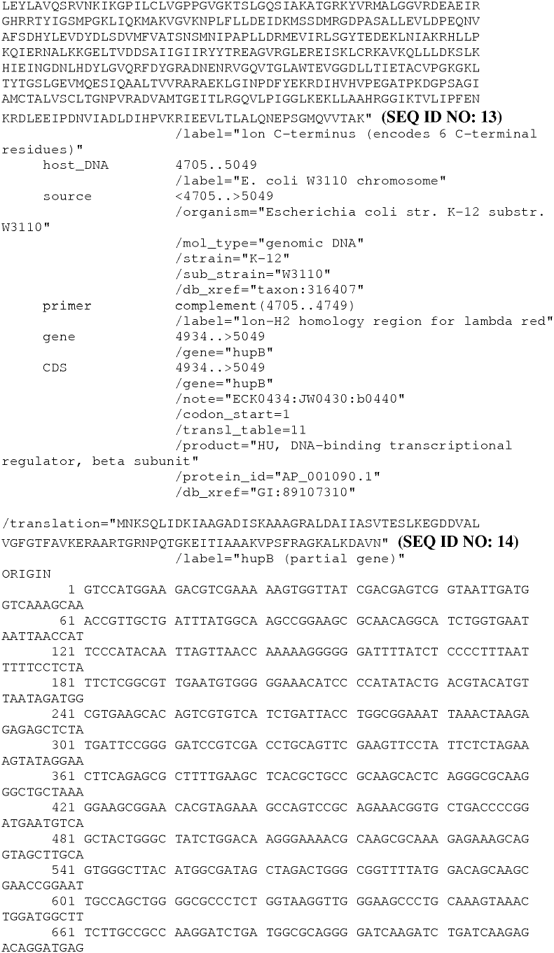

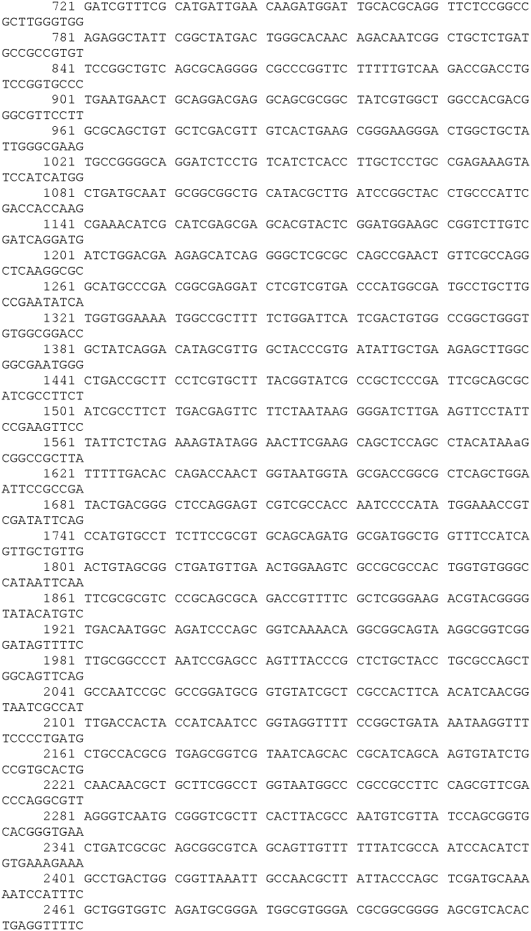

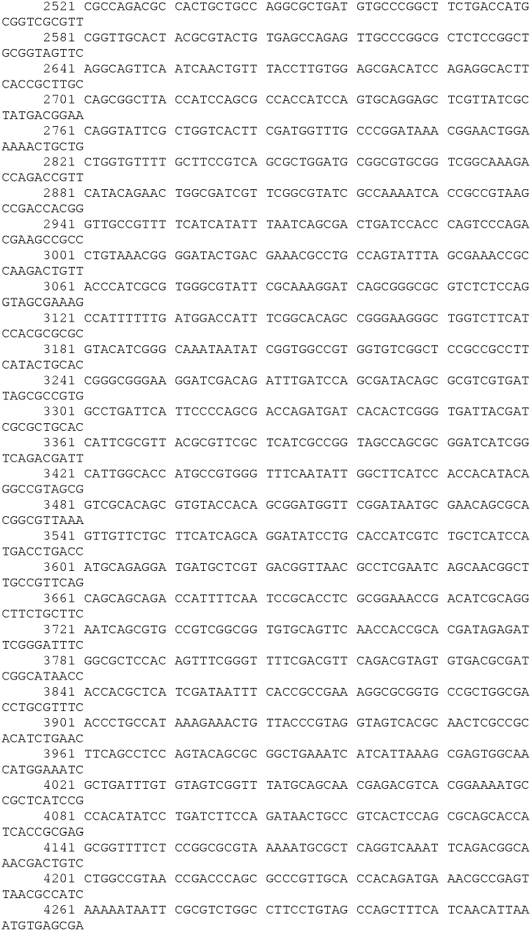

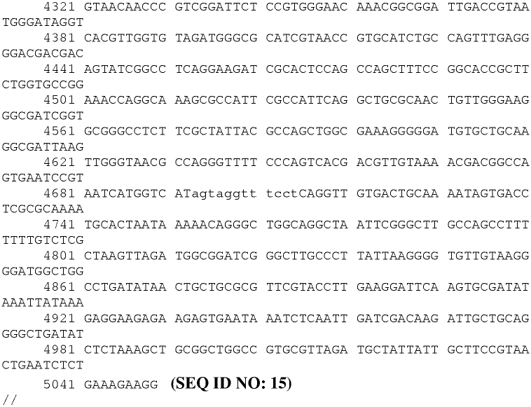

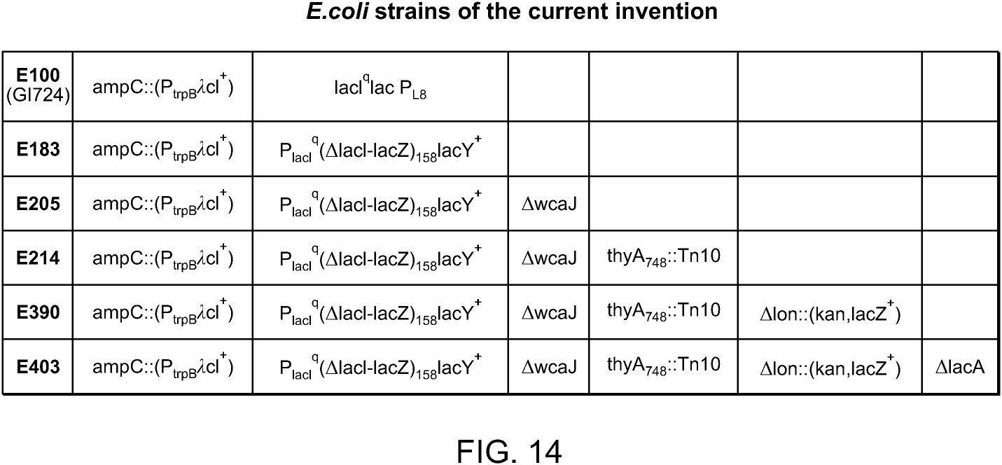

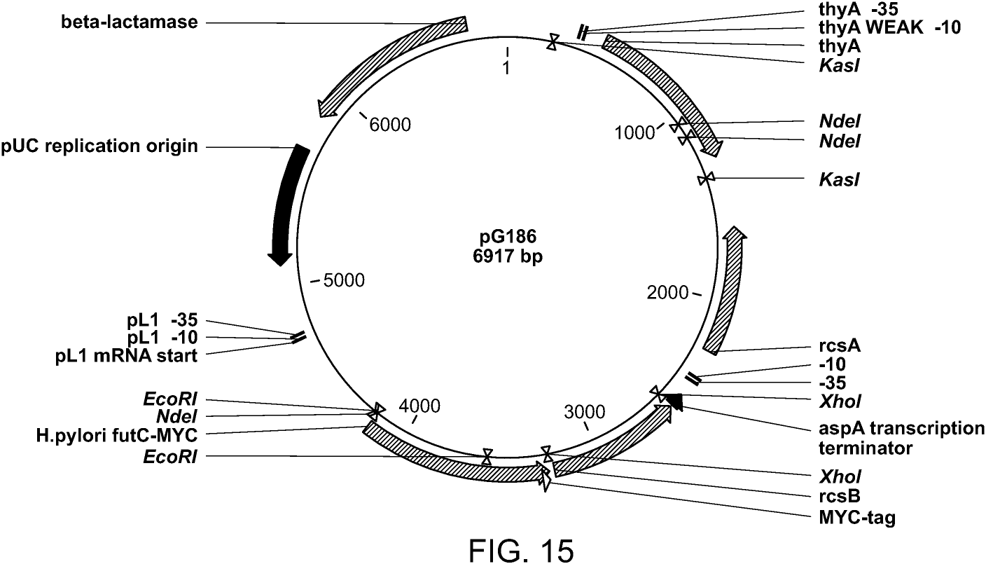

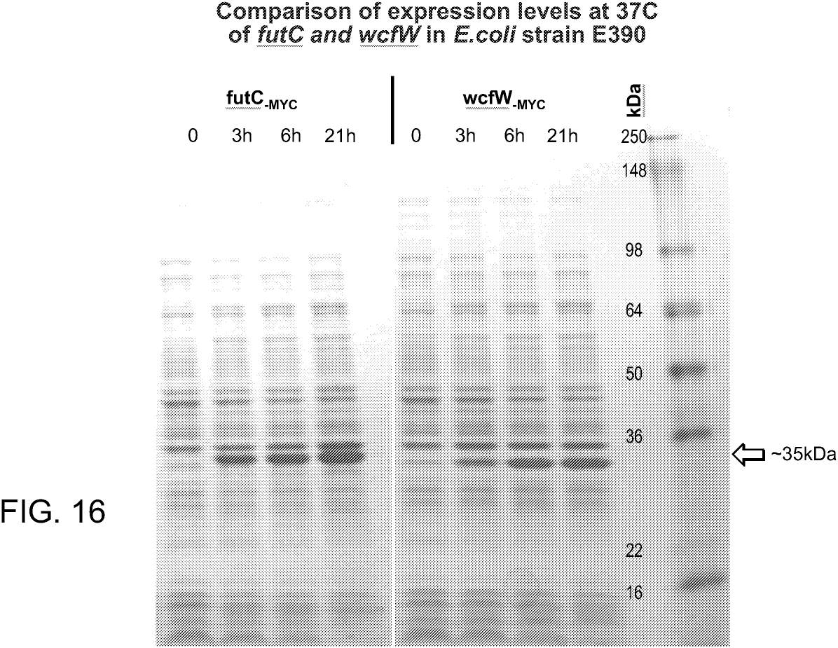

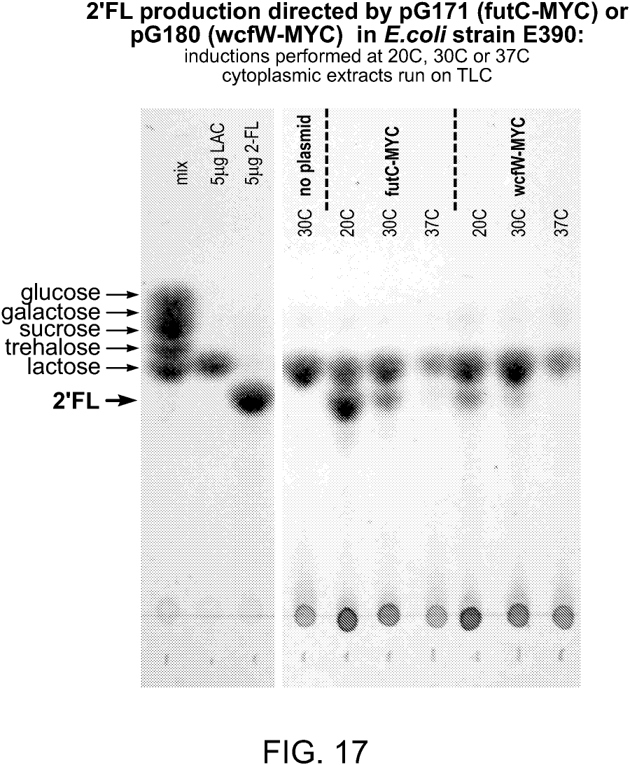

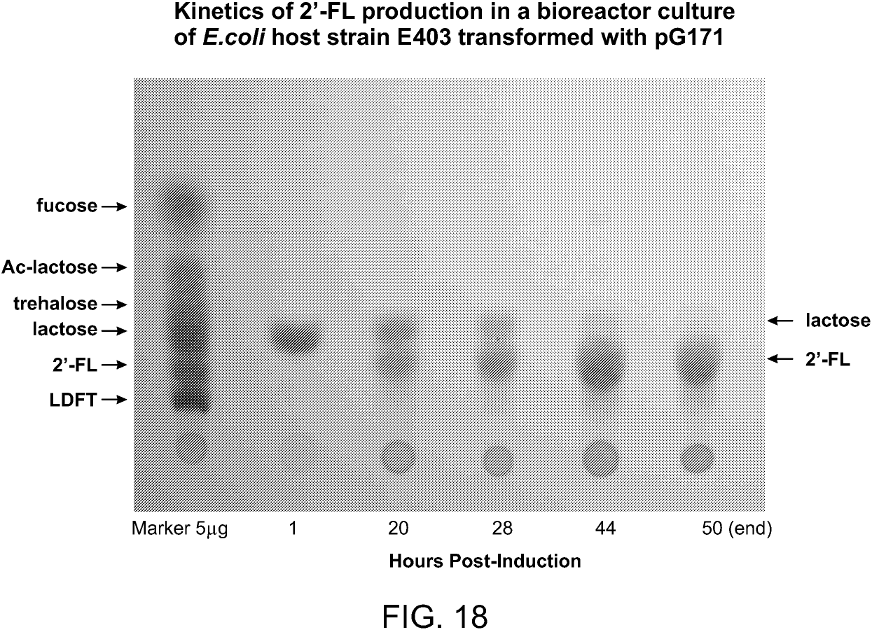

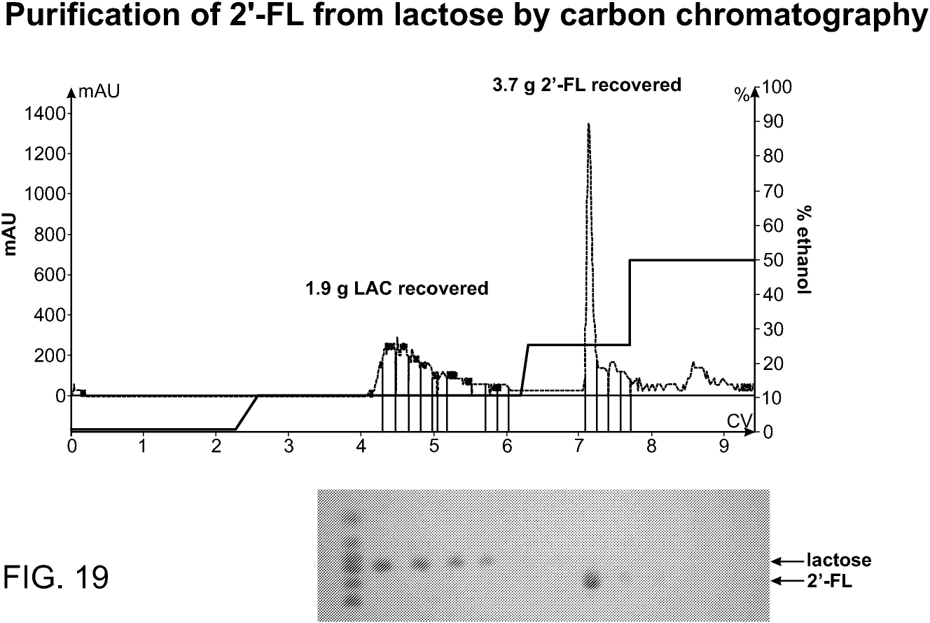

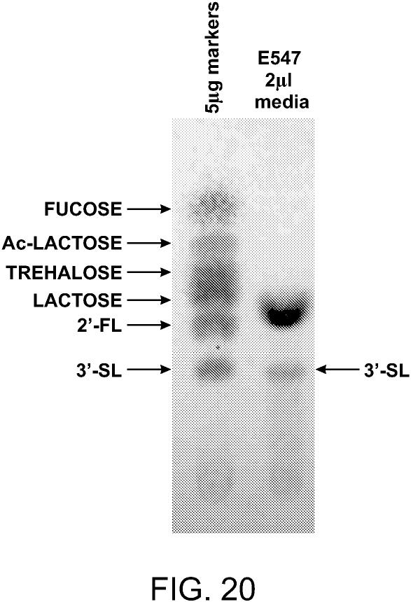

### FIELD OF THE INVENTION

The invention disclosed herein relates to non-therapeutic methods for maintaining a healthy body weight or losing body weight, in a lean subject. The invention further relates to non-therapeutic methods for increasing the concentration of short chain fatty acids (SCFA) in the distal colon of a subject. The invention also relates to 2′-fucosyllactose and resistant starch (RS) for use in the prevention of overweight or in the prevention of a condition associated with overweight in a subject; and to a composition comprising 2′-fucosyllactose (2′-FL) and RS, the use of such a composition.

### BACKGROUND

For healthy subjects, controlling body weight is often considered important for aesthetic reasons, and/or to maintain a healthy body weight and/or to lose weight.

The gut microbiota is increasingly being recognized as an important factor in fat distribution, insulin sensitivity, glucose metabolism, and lipid metabolism. Accordingly, the intestinal microbiota could play an important role in controlling body weight. One important function of the human microbiota is the fermentation of dietary fiber such as non-digestible carbohydrates. The major products of this fermentation process are the short-chain fatty acids (SCFAs) e.g. acetate, propionate and butyrate.

It is recognized that increasing the colonic, and systemically most abundant SFCAs, in particular increasing the acetate levels, may prevent diet-induced body weight gain, counteracts adiposity, improves glucose homeostasis and/or insulin sensitivity in rodents.

Furthermore, a study wherein sodium acetate was administered to the proximal or the distal colon of overweight men showed that fat oxidation and circulating plasma concentrations of the satiety hormone peptide YY (PYY) was significantly increased when acetate was administered in the distal part of the colon. In contrast, no effect on energy expenditure or substrate oxidation was seen when acetate was administered in the proximal colon (van der Beek, C. M., et al. Clinical Science 2016, 130(22), 2073-2082). In another study, distal colonic infusions of mixtures of SCFAs, all highest in acetate, increased energy expenditure, fat oxidation and plasma PYY in men (Canfora EE et al., Sci Rep. 2017, 7:2360).

This was confirmed by Bindels et al who showed that distal, but not proximal, colonic acetate infusion led to changes in metabolic markers, namely an increase in fasting fat oxidation, fasting PYY levels, postprandial glucose and insulin levels, as well as a trend towards increased fasting acetate levels. Several factors could explain the influence of the site of infusion. The proximal and distal colons differ in their microbial composition, GPR43 expression profile and drainage system, as the distal colon is drained through the general circulation and, unlike the proximal part, escapes the hepatic first-pass (Bindels et al Clin Sci. (Lond) (2016) 130 (22): 2083-2086 https://doi.org/10.1042/CS20160556.

Thus, SCFAs that are administered or generated in sufficient concentrations in the distal colon are associated with a beneficial effect on controlling body weight in a subject.

US2016/0310514A1 relates to compositions and methods for the treatment of metabolic disorders such as obesity and obesity induced pre-diabetes and type 2 diabetes, wherein the composition comprises one or more human milk oligosaccharides. It is directed at treating patients suffering from obesity; not to lean, healthy subjects.

It is desired that non-therapeutic methods be provided for increasing the concentration of SCFAs in the distal colon of a subject.

Moreover, it is desired to provide non-therapeutic methods for subjects to maintain a healthy body weight or to lose weight. For example, a desired non-therapeutic method may comprise the step of administering a compound, combination of compounds, and/or composition that increases the concentration of SCFAs in the distal colon of a subject.

In addition, it is desired that a non-therapeutic method be provided that is compatible with a normal and/or healthy diet. Preferably without negative effects on taste (of food) and/or mouth feeling. Other desired properties or effects of suitable non-therapeutic methods may include, but are not limited to, maintaining other signs of general health in the subject (e.g. maintaining a normal blood pressure, maintaining a healthy bowel movement, normal defecation, etc.), ease of preparation, ease of implementation, increasing the diversity of microbiota, and commercial availability of the compounds, combinations, and/or compositions used therein.

It is an objective of the present invention to provide a non-therapeutic method that better addresses at least one of the aforementioned desires.

### SUMMARY OF THE INVENTION

In one aspect, the present invention pertains to a non-therapeutic method for maintaining a healthy body weight or losing body weight, in a subject, characterized in that the non-therapeutic method comprises the step of administering 2′-fucosyllactose (2′-FL)and resistant starch RS to the subject; wherein the subject is a mammal with a lean weight preferably wherein the subject is a human; and wherein the method does not comprise the step of administering mother’s milk to the subject.

In another aspect, the invention relates to a non-therapeutic method for increasing the concentration of short chain fatty acids (SCFAs), preferably of acetate, in the distal colon of a subject, for prevention of diet-induced body weight gain or adiposity; or for the improvement of glucose homeostasis and/or insulin sensitivity, wherein the non-therapeutic method comprises the step of administering 2′-fucosyllactose (2′-FL)to the subject, wherein the subject is a mammal with a lean weight, preferably wherein the subject is a human; and wherein the method does not comprise the step of administering mother’s milk to the subject.

In yet another aspect, the invention relates to a composition comprising (i) 2′-FL, and (ii) resistant starch, for use in the prevention of overweight or of a condition associated with overweight in a subject, and wherein the subject is a mammal with a lean weight, preferably wherein the subject is a human.

In still another aspect, the invention pertains to a use of 2′-FL and RS for maintaining a healthy body weight or losing body weight in a subject, and wherein the subject is a mammal with a lean weight, preferably wherein the subject is a human.

In a further aspect, the invention relates to the use of a composition comprising 2′-FL, and resistant starch, for maintaining a healthy body weight or losing body weight in a subject, and wherein the subject is a mammal and has a healthy weight, preferably wherein the subject is a human.

### DETAILED DESCRIPTION OF THE INVENTION

The term “treatment”, in relation to a given disease or disorder, includes, but is not limited to, inhibiting the disease or disorder, for example, arresting the development of the disease or disorder; relieving the disease or disorder, for example, causing regression of the disease or disorder; or relieving a condition caused by or resulting from the disease or disorder, for example, relieving, preventing or treating symptoms of the disease or disorder.

The term “prevention” in relation to a given disease or disorder means preventing the onset of disease development if none had occurred, preventing the disease or disorder from occurring in a subject that may be predisposed to the disorder or disease but has not yet been diagnosed as having the disorder or disease, and/or preventing further disease/disorder development if already present.

The invention, in a broad sense, is based on the judicious insight that administering 2′-fucosyllactose with RS to a human having a BMI of less than 25 kg/m² can be used to achieve one or more of the abovementioned desires. In particular, it increases the amount of short chain fatty acids, especially acetate, in the distal colon of the subject. The combination of RS and 2′FL results in a much higher level of acetate and short chain fatty acids. In order to obtain this effect only with 2′FL, much more 2′FL would be needed, which makes such a treatment much more expensive.

It is believed that 2′-fucosyllactose in relation to the invention provides better results than other fibers known in the art.

It is known that gut microbiota may vary significantly between subjects, even between subjects of the same species, hence the gut microbiota in subjects having a healthy body weight do not necessarily exhibit the same characteristics as those of an obese subject (of the same species). Phrased differently, the effect of administering a therapeutic agent to an obese patient is not necessarily predictive for effects on a subject having a healthy body weight.

In a first aspect, the invention is directed to a non-therapeutic method for maintaining a healthy body weight or losing body weight, in a subject, characterized in that the non-therapeutic method comprises the step of administering 2′-fucosyllactose (2′-FL) and Resistant Starch (RS) to the subject; wherein the subject is a mammal with a lean weight preferably wherein the subject is a human; wherein the method does not comprise the step of administering mother’s milk to the subject.

In another aspect, the invention relates to a non-therapeutic method for increasing the concentration of short chain fatty acids (SCFAs, or SCFA for short chain fatty acid), preferably of acetate, in the distal colon of a subject, wherein the non-therapeutic method comprises the step of administering 2′-fucosyllactose (2′-FL)and RS to the subject, wherein said subject is a mammal with a lean weight preferably wherein the subject is a human; and wherein the method does not comprise the step of administering mother’s milk to the subject.

As used herein, mother’s milk is referring to milk of the same species as the subject being administered 2′-FL and RS; mother’s milk does not refer to milk of the same species enriched with 2′-FL and RS.

It is understood that sufficient 2′-FL needs to be administered in order to have the desired effect. So in one embodiment the non-therapeutic method of the invention comprises the step of administering an effective amount of 2′-fucosyllactose (2′-FL)to the subject. An effective amount is depending on the type of species. In one embodiment the amount of 2′-FL to be administered is at least 10 mg of 2′-FL per day, preferably at least 100 mg, e.g. at least 1 g, more preferably at least 2 g, e.g. at least 3 g, 4, 5, 6, 7, 8, 9, 10, 11, 12, 13, 14, or even at least 15 g per day.

The term “subject” as used herein refers to a mammalian subject (i.e. a mammal), in particular a human, that is treatable by the method of the invention. The term “subject” refers to both the male and female sex unless one sex is specifically indicated. In accordance with the invention, the subject preferably is a human subject. The human subject can be of any age, e.g. the human subject can be an infant, a juvenile, an adolescent, an adult or an elderly subject. In one embodiment of the invention, the subject is a healthy subject, in another embodiment the subject is not obese. In a further preferred embodiment the subject is a non-obese human subject.

In embodiments of the invention the human subject is at least 18 years of age, for example at least 25 years, or at least 30 years, or even at least 35 years of age. In another embodiment the human subject is at least 55 years of age, e.g. at least 60 years or at least 65 years of age. There is no particular upper limit although in practice, human subjects treated in accordance with the invention will typically be at most 100 years of age, e.g. at most 95 or at most 90 years of age. In one embodiment the human subject is between 18 and 65 years old, e.g. between 20 and 60 or between 25 and 50 years old.

The blood sugar level is indicative for diabetes. In an embodiment, the human subject has a blood sugar level during fasting of below 6.1, below 6.0, below 5.9, below 5.8, below 5.7, below 5.6 or below 5.5 mmol/L.

The 2 hour Glucose Tolerance Test (GTT) with 75 g intake is indicative for hyperglycemia. In an embodiment, the subject has a 2-hour plasma glucose level of below 7.8, below 7.6, below 7.5, below 7.4, below 7.3, below 7.2, below 7.1 or below 7.0 mmol/L as determined by the 75-g oral glucose tolerance test. It will be understood that the 2-hour plasma glucose level relates to the plasma glucose level two hours after glucose ingestion.

Whether a subject has a healthy weight can be assessed in different ways and is depending on the type of species. For humans it is generally assessed by determining the subject’s body mass index (BMI), which is defined as the body weight measured in kilograms divided by the height (in meters) squared. In a formula, BMI for a human subject, is defined as:

$$B M I \left(i n \frac{k g}{{m}^{2}}\right) = \frac{w e i g h t \left(i n k g\right)}{h e i g h t {\left(i n m\right)}^{2}}$$

A human aged 18 and above having a BMI of less than 18.5 kg/m² is considered underweight. For humans aged 18 and above, a BMI in a range of from at least 18.5 kg/m² to less than 25 kg/m² is considered a healthy body weight. A human aged 18 and above having a BMI in a range of from at least 25 kg/m² to less than 30 kg/m² is considered overweight. A. human aged 18 and above having a BMI of at least 30 kg/m² is considered obese.

The human subject aged 18 and above, in relation to the invention has a body mass index (BMI) of less than 25 kg/m². Such subject is herein also referred to as a “lean subject” or as “a person with a lean weight”. In a preferred embodiment, the human subject (aged 18 and above) in relation to the invention has a BMI in the range of from at least 18.5 kg/m² to less than 25 kg/m², i.e. said subject is considered to have a healthy body weight.

For humans aged 2 to 18 years old (children and teenagers), the BMI value as obtained using Equation 1 needs to be adjusted because girls and boys develop at different rates and have different amounts of body fat at different ages. For this reason, BMI measurements during childhood and adolescence take age and sex into consideration. For children and teenagers, aged 2 to 18, a healthy body weight is defined as a BMI value in accordance with the values listed in Table 1.

TABLE 1

definition of healthy body weight BMI values [in kg/m²] for human subjects

| Age | Male BMI (healthy body weight) | Female BMI (Healthy body weight) |
|---|---|---|
| 2 | 15.14 - 18.40 | 14.83-18.01 |
| 3 | 14.74 - 17.88 | 14.47 - 17.55 |
| 4 | 14.43 - 17.54 | 14.19-17.27 |
| 5 | 14.21 - 17.41 | 13.94-17.14 |
| 6 | 14.07 - 17.54 | 13.82- 17.33 |
| 7 | 14.04 - 17.91 | 13.86 - 17.74 |
| 8 | 14.15 - 18.43 | 14.02 - 18.34 |
| 9 | 14.44 - 19.09 | 14.28-19.06 |
| 10 | 14.64 - 19.83 | 14.61 - 19.85 |
| 11 | 14.97 - 20.54 | 15.05 - 20.73 |
| 12 | 15.35 - 21.21 | 15.62-21.67 |
| 13 | 15.84 -21.90 | 16.26 - 22.57 |
| 14 | 16.41 - 22.61 | 16.88 - 23.33 |
| 15 | 16.98 - 23.28 | 17.45 - 23.93 |
| 16 | 17.54 -23.89 | 17.91 - 24.36 |
| 17 | 18.05 - 24.45 | 18.25 - 24.69 |
| ≥18 | 18.50 - 24.99 | 18.50 - 24.99 |

A child or teenager with a “lean body weight” or a “lean weight” is defined as a child or teenager with a BMI value corresponding to a “healthy body weight or a lower BMI value as defined in Table 1 for that sex and age”.

As used herein a “lean weight” for subjects not being a human, is defined as the subject having a healthy weight or having a mass lower than a healthy weight. A healthy weight is defined differently for different species and typical values per species are well known in the art. A “lean weight” mean the subject is not obese.

Typically, the non-therapeutic methods of the invention are carried out for non-medical reasons, e.g., for cosmetic purposes. Hence, in an embodiment of the invention the non-therapeutic method is for controlling weight, losing weight, reducing weight, preventing weight gain, limiting weight gain, inducing weight loss, increasing weight loss, managing weight and/or maintaining a healthy weight, in a subject as defined herein.

In a particular embodiment of the invention, the non-therapeutic method is a method of losing weight within a predetermined interval, e.g. to lose weight within 12 months, within 6 months, within 4 months, within 3 months, within 2 months, within 1 month, within 4 weeks, within 3 weeks, within 2 weeks or within 1 week.

As used herein, an increase in the concentration of short chain fatty acids (SCFAs), in the distal colon of a subject refers to an increase of the combined level of acetate, propionate, and butyrate. These levels may be determined using the TIM-2 model system as known in the art and as described elsewhere herein (e.g. see Examples). An increase of SCFAs is defined as an increase of SCFAs of at least 10%, preferably at least 20% more preferably at least 40%, even more preferably at least 50% as determined by measuring the total amount of SCFAs produced in the last 16 hours of the experiment (representing the amounts produced in the distal colon) using the TIM-2 model as compared to a reference diet.

As used herein, “distal colon” in humans refers to the descending colon (the left side of the colon) and the sigmoid colon (the S-shaped section of the colon that connects to the rectum), or to the corresponding part of the colon in other mammals.

Both the above-mentioned effects on body weight and on SCFA(s) levels may be obtained simultaneously in the method of the invention. Hence, in one embodiment the invention relates to a non-therapeutic method for i) maintaining a healthy body weight or losing body weight, in a subject, and ii) increasing the concentration of SCFA, preferably of acetate, in the distal colon of a subject; characterized in that the non-therapeutic method comprises the step of administering 2′-fucosyllactose (2′-FL) and RS to the subject; wherein the method does not comprise the step of administering mother’s milk to the subject; wherein the subject is a mammal with a lean weight preferably wherein the subject is a human wherein the subject is a human with a lean body weight.

As used herein, “short chain fatty acids” (SCFAs) and “short chain fatty acid” (SCFA) may be used interchanged to refer to acetate, propionate and butyrate.

In another embodiment, the subject being administered the 2′-FL and RS is a human aged 2 or more, with a body mass index (BMI) equal to or less than the upper value indicated in Table 1 for a healthy weight for the age and sex of the subject.

Administration of 2′-FL and RS is preferably done orally as this is normal way to administer food.

In another embodiment, the non-therapeutic method of the invention comprises the step of administering 2′-fucosyllactose (2′-FL) and comprises the step of administering resistant starch. In yet another embodiment the non-therapeutic method of the invention comprises the step of simultaneously administering 2′-FL and resistant starch. In still another embodiment, the non-therapeutic method of the invention comprises the step of administering 2′-FL and resistant starch, wherein the 2′-FL and resistant starch are administered sequentially i.e. first 2′-FL and then resistant starch or first resistant starch and then 2′-FL,

In a favorable embodiment, the non-therapeutic method of the invention comprises a step of administering 2′-fucosyllactose, wherein 2′-fucosyllactose is comprised in a composition additionally comprising resistant starch. Such a composition may comprise further components such as vitamins, odors and color flavorings.

It is believed that the combination of 2′-FL with resistant starch provides an improvement in the release of SCFAs in the distal part of the colon. As will be understood by those skilled in the art, the benefits of the present invention may also be accomplished by sequential or simultaneous administration of 2′-FL, on the one hand, and resistant starch on the other hand, possibly in the form of two separate compositions each comprising one of these components. Such methods, uses and treatment are also within the scope of the present invention. It is, however, contemplated that the co-administration of the combination as part of a single composition of product is particularly advantageous.

Without wishing to be bound by theory, the present inventors currently believe that the colonic microbiota first ferment the resistant starch as an energy source. Thus, it is believed that (more of) the 2′-FL can reach the distal colon where it is fermented, by other gut microbes which produce the SCFAs (in particular) acetate at that region, whereby the acetate may exert beneficial effects locally and/or may enter the systemic circulation, consequently leading to its metabolic effects.

2′-Fucosyllactose (2′-FL) is an oligosaccharide, more precisely, a fucosylated, neutral trisaccharide composed of L-fucose, D-galactose, and D-glucose units, linked Fuc(al-2)Gal(B1-4)Glc; CAS Nr 41263-94-9. It is the most prevalent human milk oligosaccharide (HMO) naturally present in human breast milk, making up about 30% of all of HMOs. HMOs are non-digestible carbohydrates and are the third most abundant component in human milk after lactose and fat. More than 200 different oligosaccharides have currently been identified in human milk. It has been suggested in clinical trials that 2′-FL plays a key role in protecting and promoting the health of newborn infants, particularly in respect to the immune system. It has been shown that the addition of 2′-FL to infant formula is safe and well-tolerated. In addition, 2′-FL is safe and well-tolerated for all other age groups, especially for adults.

HMOs can be obtained using methods known to those of skill in the art. For example, HMOs can be purified from human milk. Individual HMOs can be further separated using methods known in the art such as capillary electrophoresis, HPLC (e.g., high-performance anion-exchange chromatography with pulsed amperometric detection; HPAEC-PAD), and thin layer chromatography. See, e.g., U.S. Pat. Application No. 2009/0098240. Alternately, enzymatic methods can be used to synthesize HMOs. Another method to manufacture HMO’s is via biosynthesis in engineered bacteria. For example, a method of preparing 2′-FL is disclosed in WO 2012/112777. Alternatively, 2′-Fl, is commercially available e.g. from FrieslandCampina, or others.

2′-FL is a dietary fiber. For most humans, there is a maximum amount of dietary fiber that can be consumed on a daily basis. The amount of 2′-FL in the composition as used in the invention also depends on the body weight (i.e. mass) of the subject. So, in one embodiment the amount of 2′-Fl, in the composition as used in the invention is more than 0.1 gram. In another embodiment, it is in the range of from 0.1 to 30 gram, preferably in a range of from 0.5 to 25 gram, more preferably in a range of from 1 to 20 gram, most preferably in a range of from 2 to 10 gram.

LNnT may be considered as a dietary fiber. In one embodiment the composition for use according to the invention does not comprise lacto-N-neotetraose (LNnT).

Resistant starches may be used in the invention. Starches are polysaccharides composed of a number of a-D-glucose molecules linked with (1-4) and/or (1-6) linkages. Starch consists of two main structural components, the amylose, which is essentially a linear polymer in which ci-D-glucosc-,, residues are -(1-4) linked, typically constituting 15% to 20% in most starches, and amylopectin, which is a branched molecule with (1-4) and D-(1-6) linkages between the a-D-glucose units, and is the major component of most starches, Starches can be classified according to their behavior when incubated with enzymes without prior exposure to dispersing agents. According to this system starches can be classified as rapidly digestible starch (RDS), slowly digestible starch (SDS) and resistant starch (RS). The term “resistant starch” was first used by Englyst et al. in 1982 to describe a small fraction of starch that was resistant to hydrolysis by exhaustive a-amylase and pullulanase treatment in vitro. RS was the starch not hydrolyzed after 1 min of incubation. However, because starch reaching the large intestine may be more or less fermented by the gut microflora, RS is now herein defined as that fraction of dietary starch, which escapes digestion in the small intestine. It is measured chemically as the difference between total starch (TS) obtained from a homogenized and chemically treated sample and the sum of RDS and SDS, generated from non-homogenized food samples by enzyme digestion (RS = TS -(RDS + SDS)). Resistant starch is usually further classified as RS1, RS2, RS3 or RS4.

RS1 refers to starch that is physically inaccessible as it is locked within cell walls of botanical substances and therefore qualifies as resistant starch. The RS1 type of resistant starch is, for example, found in partially milled grains, seeds, and legumes. RS1 is heat stable in most normal cooking operations and enables its use as an ingredient in a wide variety of conventional foods.

RS2 refers to native resistant starch which is a component of starch granules such as those found in bananas (especially green bananas) and raw potatoes. Bananas and raw potatoes have relatively low gelatinization temperatures, typically on the order of about 60° C. to about 80° C., which often presents substantial problems in the formulation of food products. Intragranular polymeric, rearrangements that lead to an increased granular resistance to amylase digestion are also included in this category. This increased resistance could be the result of heat and/or moisture treatments or annealing of the intact granule. It is measured chemically as the difference between the glucose released by the enzyme digestion of a boiled homogenized food sample and that from an unboiled, non-homogenized food sample. In raw starch granules, starch is tightly packed in a radial pattern and is relatively dehydrated. This compact structure limits the accessibility of digestive enzymes, various amylases, and accounts for the resistant nature of RS2 such as, ungelatinized starch. In the diet, raw starch is consumed in foods like banana. RS1 and RS2 represent residues of starch forms, which are digested very slowly and incompletely in the small intestine.

RS3 refers to retrograded non-granular starch or crystalline non-granular starch, such as starch found in cooked and cooled potatoes, bread crusts, and cereals (cornflakes, for example) and starch pastes that have been extensively processed (by repeated cooking and cooling). It is measured chemically as the fraction, which resists both dispersion by boiling and enzyme digestion. It can only be dispersed with KOH or dimethyl sulphoxide. RS3 is entirely resistant to digestion by pancreatic amylases.

RS4 refers to specific starches that have been chemically modified and/or re-polymerized (which may include molecular weight reduction), such as ethers, esters, and cross-bonded starches, as well as chain linkage altered dextrins, pyrodextrins, and maltodextrins.

The resistant starch that is utilized in the present invention may be any resistant starch, such as any of the RS1, RS2, RS3, or RS4 resistant starches or any combination of two or more thereof. Preferably, the resistant starch that is utilized in the present invention is selected from RS2, RS3 and RS4, more preferably from RS2 and RS3. Most preferably the resistant starch is RS2.

In accordance with the invention, resistant starch can suitably be used that is derived from sources such as corn, wheat, rice, legume, pea, banana, barley, triticale, sorghum, milo, cassava, oat, potato, tapioca, sago, ocarina, etc. In a preferred embodiment of the invention, the resistant starch is derived from corn, potato or banana, more preferably from corn or potato. In an embodiment of the invention, the resistant starch is selected from the group comprising resistant waxy maize starch; resistant regular or normal maize starch; resistant wheat starch; resistant rice starch; resistant legume, pea or pulse starch; resistant barley starch; resistant triticale starch; resistant sorghum starch; resistant milo starch; resistant cassava starch; resistant banana starch, resistant oat starch; resistant potato starch; resistant tapioca starch; and resistant sago starch, more preferably from the group comprising resistant corn starch, resistant potato starch and resistant banana starch, most preferably from resistant corn starch and resistant potato starch.

In embodiments of the invention, the resistant starch is characterized by its specific amylose content. In one embodiment of the invention the resistant starch is characterized by a high amylose content, such as an amylose content, based on the total dry weight of the starch, of at least 35 wt.%, e.g. at least 40 wt.%, at least 45 wt.%), at least 50 wt.%, or at least 55 wt.%. There is no particular upper limit, although practically the resistant starch will have an amylose content, based on the total dry weight of the starch, of less than 75 wt.%, less than 70 wt.%, less than 65 wt.% or less than 60 wt.%. Currently, a high-amylose corn starch with the amylose content of up 70 wt.% is available.

Based on X-ray diffraction patterns, starches can be classified as type A, type B and type C. The type A structure has amylopectin of chain lengths of 23 to 29 glucose units. The hydrogen bonding between the hydroxyl groups of the chains of amylopectin molecules results in the formation of outer double helical structure. In between these micelles, linear chains of amylose moieties are packed by forming hydrogen bonds with outer linear chains of amylopectin. This pattern is very common in cereals. The type B structure consists of amylopectin of chain lengths of 30 to 44 glucose molecules with water inter-spread. This is the usual pattern of starches in raw potato and banana. The type C, structure is made up of amylopectin of chain lengths of 26 to 29 glucose molecules, a combination of type A and type B, which is typical of peas and beans.

One of the causes of resistance to enzymes is the crystallinity of native type B starch granules. In accordance with the present invention, it is preferred that the resistant starch is a type 13 (resistant) starch. In embodiments of the invention, the resistant starch is characterized by its specific granule size distribution. In an embodiment of the invention, the resistant starch is characterized by a volume-weighted mean diameter D[4,3] of at least 5 µm, at least 7.5 µm, at least 10 µm, at least 12.5 µm, at least 15 µm, at least 17.5 µm, at least 20 µm, at least 22.5 µm or at least 25 µm. In an embodiment of the invention, the resistant starch is characterized by a volume-weighted mean diameter D[4,3] of less than 150 µm, less than 100 µm less than 75 µm, less than 50 µm, less than 40 µm, less than 35 µm, less than 30 µm, or less than 25 µm. A volume-weighted mean diameter D[4,3] may for example be determined using a Malvern Mastersizer system.

In embodiments of the invention resistant starch is a high amylose maize starch. Some suitable, though non-exhaustive, examples of high amylose starch are the HIMAIZE™ high amylose starches (ex Ingredion, Westchester, USA), such as HIMAIZE ® 260.

In embodiments of the invention, the resistant starch is granular potato starch. A suitable example thereof includes type 2 resistant starch Potato Starch Food Grade Quality, which is derived from potatoes; it is a granulated, light beige powder, is intended for use in food and has a GRAS (Generally Recognized As Safe) status (ex AVEBE, Veendam, The Netherlands).

In embodiments of the invention, the resistant starch is resistant tapioca starch. A suitable example thereof includes C* Actistar 11700 (ex Cerestar, France).

The non-therapeutic methods and treatments as described herein comprise the administration to the subject of 2′-Fl, with resistant starch, all as described herein, in an effective amount. Typically, the non-therapeutic methods entail the administration of the 2′-FL in unit dose form. The resistant starch is typically also administered in unit dose form. Such a unit dose may take any form, including the form of an alimentary product comprising the 2′-FL and the resistant starch, wherein the alimentary product is provided in the form of a single serving, each serving comprising the 2′-FL and the resistant starch in unit dose amount. A single serving may be individually packaged. A unit dose is herein defined as the amount of an ingredient administered to a subject in a single dose. Depending on the unit dose, one or more single servings may be administered during a day.

The term “single serving” as used herein refers to a certain quantity and/or size of the product that is adequate for consumption as a single portion for a single person. Such products may be in a form that is ready-to-eat or ready-to-consume or it may be in a form that requires further processing, such as heating or addition of a quantity of hot or cold water.

In one embodiment, the composition as used in the method of the invention is a food product, preferably in the form of a single serving, comprising 2′-Fl, and resistant starch. A single serving may be individually packaged.

In a preferred embodiment, the unit dose amount of the 2′-FL is at least 0.5 gram, at least 1 gram, at least 1.5 gram, at least 2 gram, at least 2.5 gram, at least 3 gram, at least 3.5 gram, or at least 4 gram.

In another embodiment, the unit dose amount of the 2′-FL is at most 25 gram, e.g. at most 20 gram, at most 15 gram, at most 12.5 gram, at most 10 gram, at most 9 gram, at most 8 gram, at most 7 gram, at most 6 gram or even at most 5 gram.

In yet another embodiment, the unit dose amount of the 2′-FL is in a range of from 0.5-10 gram, g, preferably 1-8 gram, more preferably 2-4 gram. In an embodiment, the unit dose amount of the 2′-F1-i is 3-8 gram, preferably 3- 7 gram, more preferably 3-6 gram.

In still another embodiment, the unit dose amount of the resistant starch is at least 0.5 gram, e.g. at least 1 gram, at least 1.5 gram, at least 2 gram, at least 2.5 gram, at least 3 gram, at least 3.5 gram, or at least 4 gram. In one embodiment, the unit dose amount of the resistant starch is at most 2.5 gram, e.g. at most 20 gram, at most 15 gram, at most 12.5 gram, at most 10 gram, at most 9 gram, at most 8 gram, at most 7 gram, at most 6 gram or at most 5 gram. In a particular embodiment, the unit dose amount of the resistant starch is 0.5-10 gram.

In still another embodiment, the unit dose amount of the 2′-FL is in a range of from 0.5-15 gram and the unit, dose amount of the resistant starch is 0.5-1 5 gram, preferably the unit dose amount of the 2′-Fl-, is in a range of from 1-10 gram and the unit dose amount of the resistant starch is 1-10 gram, more preferably, the unit dose amount of the 2′-FL is in a range of from 2-8 gram and the unit dose amount of the resistant starch is 1-8 gram.

In another embodiment the composition used in the method of the invention comprises an amount of 2′-fiicosyl lactose of at least 0.5 gram e.g. at least 1.0 gram, such as at least 2.0, 4.0, 6.0 8.0, 10.0, 12, or even at least 15 gram. In still another embodiment the composition further comprises an amount of resistant starch of at least 0.5 gram, e.g. at least 1.0 gram, such as at least 2.0, 4.0, 6.0 8.0, 10.0, 12, or even at least 15 gram.

As will be understood by those skilled in the art, based on the present teachings, the administration in the method of the invention is preferably done orally, in the form of a single composition or, alternatively 2′ - FL and resistant starch are administered in two compositions being administered simultaneously or sequentially. In embodiments wherein the 2′-FL, and the optional resistant starch are administered sequentially, it is preferred that the time in between the administration of the respective compositions is at most 5 hours, preferably at most 4 hours, at most 3 hours, at most 2 hours, at most 1 hour, at most 30 minutes, at most 20 minutes, at most 15 minutes, at most 10 minutes or at most 5 minutes. In case of a sequential administration, it is preferred that the resistant starch is administered prior to the administration of 2′-FL.

The compositions or unit doses of the 2′-FL and resistant starch are preferably administered at least once a week, preferably at least once every 3 days, at least once every other day, at least once daily. In preferred embodiments of the invention, the non-therapeutic methods comprise the daily administration of unit doses of the 2′-FL.., or of 2′ -F^{L} and resistant starch, preferably once a day, twice a day, three times a day or four times a day, more preferably once or twice a day, most preferably once a day.

In accordance with the invention, the non-therapeutic methods as defined herein, are preferably continued for a period of at least two weeks, more preferably at least 3 weeks, at least 4 weeks, at least 1 month, at least two months, at least three months, at least 4 months, at least 5 months, or at least 6 months.

In an embodiment of the invention, the non-therapeutic methods comprise the administration of the 2′-FL in an average amount of 0.5-32 gram per day, preferably in an average amount of 1-24 gram per day, more preferably in an average amount of 4-16 gram per day, e.g. approximately 12 gram per day, preferably over a period of at least 2 weeks, preferably at least 3 weeks, at least 4 weeks, at least 1 month, at least 2 months, at least 3 months, at least 4 months, at least 5 months, or at least 6 months.

In other embodiments, the non-therapeutic methods as defined herein comprise the administration of resistant starch in an average amount of 0.5-32 gram per day, preferably in an average amount of 2-24 gram per day, more preferably in an average amount of 3-16 gram per day, more preferably 4-12 gram per day, e.g. approximately 7.5 gram per day, preferably over a period of at least 2 weeks, preferably at least 3 weeks, at least 4 weeks, at least 1 month, at least 2 months, at least three months, at least 4 months, at least 5 months, or at least 6 months.

Infurther embodiments, 2′-FL is comprised in a composition. When 2′-FL.. is comprised in a composition, 2′-FL is preferably present in an amount of at least 5 wt.%, at least 10 wt.%, at least 20 wt.%, at least 30 wt.%, at least 40 wt.%, at least 50 wt. %, at least 60 wt.%, at least 70 wt.%, at least 80 wt.%, at least 90 wt.% or at least 95 wt.% as compared to the total weight of the composition. In preferred embodiments, 2′-FL is present in a range of from 5 wt. % to 95 wt.%, more preferably in a range of from 10 wt. % to 90 wt. %, even more preferably in a range of from 20 wt.% to 80 wt.%, more preferably still in a range of from 25 wt.% to 75 wt.%, most preferably in a range of from 30 wt.% to 60 wt.% as compared to the total weight of the composition.

In a particularly favorable embodiment, the composition comprising 2′-FL and resistant starch. Therein, 2′-FL is present in the wt%-amounts as defined above, and resistant starch is present in an amount of at least 5 wt. % as compared to the total weight of the composition. Preferably, 2′-FL is present in the composition in an amount of at least 5 wt. % and resistant starch is present in an amount of at least 5 wt.% as compared to the total weight of the composition. More preferably, resistant starch is present in the composition in an amount of at least 10 wt. %), at least 20 wt.%, at least 30 wt.%, at least 40 wt. %J, at least 50 wt. %, at least 60 wt. %, at least 70 wt. %, at least 80 wt. %, at least 90 wt. %, or at least 95 wt. % as compared to the total weight of the composition.

In preferred embodiments, in the composition comprising 2′-F′I, and resistant starch, 2′-FL and resistant starch are provided in a weight ratio in a range of from 0.5:10 to 10:0.5, preferably in a weight ratio in a range of from 1:8 to 8:1, more preferably in a range of from 2:6 to 6:2, most preferably in a range of from 3:5 to 5:3. In a preferred embodiment, 2′ - FL and resistant starch are provided in the composition in a weight ratio of about 1:1.

In still another embodiment, the combined amount of 2′-FL and resistant starch in the composition as used in the non-therapeutic method of the invention is at most 30 gram, preferably at most 25 gram, more preferably at most 20 gram, most preferably at most 15 gram. The maximum combined amount of 2′-FL and resistant starch as referred to in this embodiment relate to the maximum amount an adult human subject can consume on a daily basis without disturbing a normal defecation. Generally, higher amounts result in diarrhea or liquid / watery bowel movements.

In another embodiment, the composition consists essentially of 2′-FL and resistant starch in amounts or in a ratio as defined above.

In still another embodiment of the non-therapeutic methods of the invention, 2′-FL is comprised in a food product preferably selected from the group comprising dairy product e.g. as milk-product, milkshake, chocolate milk, yoghurt, pudding, cream, cheese, ice cream etc.; bar, such as nutritional bar, energy bar, snack bar, cereal bar, bar for diabetics etc.; liquid product, such as nutritional drink, diet drink, liquid meal replacers, sports drink and other fortified beverages; dessert-type product, such as pudding, yoghurt; savory snack, such as chips, tortillas, puffed and baked snacks, crackers, pretzels; savory biscuit, bakery products, such as muffins, cakes, biscuits; pasta, such as spaghetti; and food supplements e.g. pills, capsules, or dry powder. Food supplements may be ready for consumption or may need to be dissolved in a liquid like water. The product in dry powder form may be accompanied with a device, such as a spoon, to measure the desired amount of the powder (e.g. daily or unit dose). Food supplements may further comprise other ingredients commonly used in food supplements such as vitamins, minerals, salts, etc. The food product is preferably selected from the group consisting of dairy product, liquid product, and food supplement.

The composition as defined herein or the food product as defined herein may be provided in a jar, bottle, sachet, carton, wrapping, and the like.

In preferred embodiments, the composition or food product as used in the method of the invention comprises 2′-FL and optionally resistant starch as defined herein, in an amount of at least 10 wt.%, at least 20 wt. %, at least 30 wt.%, at least 40 wt.%, at least 50 wt.%, at least 60 wt.%, at least 70 wt.%, at least 80 wt.%, at least 90 wt. %, or 100 wt. % as compared to the total weight of the composition or food product.

In one embodiment, the food or dietetic product is in the form of single servings, preferably each serving comprising 0.5-25 gram of 2′-FL, more preferably 1-20 gram of 2′-FL; more preferably each serving comprising 0.5-15 gram of 2′-Fl, and 0.5-10 gram of resistant starch, even more preferably each serving comprising 0.5-10 gram, such as 2-8 gram, of 2′-FL, and 0.5-10 gram of resistant starch; particularly preferably each serving comprising 0.5-10 gram, such as 2-8 gram, of 2′-FL, and 1-6 grams, of resistant starch. Optionally each single serving may be individually packaged.

The amount of 2′-FL or of the composition comprising 2′-FL and resistant starch as defined herein contained in a specific food product in relation to the invention depends on the kind of food product, in particular its size and composition, as well as on the frequency and amount in which the product is or is supposed to be consumed.

In a particularly preferred embodiment several single servings of the food product or dietetic product to be used in the method of the invention may be packed in a container to provide sufficient single servings for a number of days e.g. a week or month.

In another aspect, the invention relates to 2′-fucosyllactose for use in the prevention of overweight or of conditions associated with overweight in a subject wherein the subject is a mammal with a lean weight, preferably wherein the subject is a human, more preferably wherein the subject is a human aged 2 or more, with a body mass index (BMI) equal to or less than the maximum value indicated in Table 1 for the age and sex of the subject.

In one aspect the invention relates to the use of 2′-fucosyllactose in the prevention of overweight or of conditions associated with overweight in a subject wherein the subject is a mammal with a lean weight, preferably wherein the subject is a human, more preferably wherein the subject is a human aged 2 or more, with a body mass index (BMI) equal to or less than the maximum value indicated in Table 1 for the age and sex of the subject.

In still a further aspect, the invention relates to a composition comprising (i) 2′-fucosyllactose, and (ii) resistant starch, for use in the prevention of overweight or of conditions associated with overweight in a subject, wherein the subject is a mammal with a lean weight, preferably wherein the subject is a human, more preferably wherein the subject is a human aged 2 or more, with a body mass index (BMI) equal to or less than the maximum value indicated in Table 1 for the age and sex of the subject. In yet another aspect the invention relates to the use of such a composition for maintaining a healthy body weight or losing body weight in a subject wherein the subject is a mammal with a lean weight preferably wherein the subject is a human. In yet another aspect the invention relates to the use of such a composition for increasing the concentration of short chain fatty acid (SCFA), preferably of acetate in the distal colon of the subject, wherein the subject is a mammal with a lean weight preferably wherein the subject is a human.

The therapeutic use of compositions comprising 2′-FL and resistant starch according to the invention, will typically rely on the use of the same compounds, compositions and products, and amounts thereof, as well as the same routes of administration and the same dosage regimens as defined herein above in relation to non-therapeutic methods.

As already indicated herein before, the present invention resides in one aspect in the finding that the (oral) administration of the combination of the invention to a subject results in an increase in the SCFA levels in the distal part of the colon. Without wishing to be bound by theory, it is understood that such an increase in SCFA levels is beneficial to the prevention of a variety of diseases or conditions, including, in particular, overweight and overweight-related diseases and conditions.

Because overweight is associated with the onset or progression of other diseases, the combinations and methods of the invention are further useful in methods of reducing complications associated with overweight including vascular disease, hypertension, insulin resistance, diabetes and musculoskeletal diseases. The present invention, in various embodiments, provides combinations for use in methods of preventing these overweight-associated diseases or conditions in a subject as defined herein.

The invention also pertains to a method for preventing overweight and/or a condition associated with overweight as defined herein, said method comprising the step of administering 2′-FL and RS to a subject as defined herein. In one embodiment, in the method for preventing overweight and/or a condition associated with overweight, 2′-FL is comprised in a composition further comprising resistant starch. In the method for preventing overweight and/or a condition associated with overweight according to the invention, 2′-FL and resistant starch, are used in the same amounts, ratios, dosage regimens, dietetic products, etc. as defined herein.

In still another aspect, the invention relates to the use of 2′-FL and RS in the manufacture of a medicament for the treatment of overweight or of conditions associated with overweight.

In yet another aspect the invention relates to a composition comprising at least 5 wt% of 2′-FL and at least 5 wt% of resistant starch wherein the wt% is determined on total solids of the composition. Preferably the total amount of insoluble, non-digestible carbohydrates in this aspect of the invention is between 10 and 100% to 95 wt.% based on total solids. More preferably the total amount of 2′-FL and resistant starch in this aspect of the invention is between 25 and 75 wt.% based on total solids.

The composition of the invention or the composition as used in the use or method of the invention may further comprise other ingredients which may contribute to the general well-being of the subject. In one embodiment, these other ingredients include probiotics, in particular probiotics which can help a subject to lose weight such as Lactobacillus fermentum, Lactobacillus amylovorus, or Lactobacillus gasseri. In another embodiment, such other ingredients may comprise one or more ingredients selected from the group of inulin, oligofructans (i.e. fructo-oligosaccharides (FOS), xylans (i.e. xylo-oligosaccharides (XOS), mannans (i.e. mannan-oligosaccharides (MOS), beta-glucans (i.e. beta 1-3, beta 1-4 and / or 1-6 beta-glucans), pectins, vitamins, and galacto-oligosaccharides (GOS). Preferably such other ingredients comprise one or more ingredients from the group of inulin and oligofructans (i.e. fructo-oligosaccharides (FOS)).

Another aspect of the invention relates to a method of treating a human suffering from undesired weight gain, by administering an effective amount of 2′-FL, preferably by administering an effective amount of 2′-FL and resistant starch. The amount of 2′-FL and resistant starch per day, dosage, unit or serving is as defined elsewhere herein. In one embodiment of this aspect, the subject has a lean body weight, preferably, the subject is aged 18 or older.

Another aspect of the invention relates to a method of treatment a human desiring to lose body weight or maintaining a healthy body weight, by administering an effective amount 2′-FL, preferably by administering an effective amount of 2′-FL and resistant starch. The amount of 2′-FL and resistant starch is as defined elsewhere herein. In one embodiment of this aspect, the subject has a lean body weight, preferably, the subject is aged 18 or older.

Except in the examples, or where otherwise expressly indicated, all numerical quantities in this description indicating amounts of material or conditions of reaction and/or use are to be understood as modified by the word “about” in describing the broadest scope of the invention. Practicing the invention within the numerical limits stated is generally preferred. Also, unless expressly stated to the contrary: percent, “parts of,” and ratio values are by weight; the description of a group or class of materials as suitable or preferred for a given purpose in connection with the invention implies that mixtures of any two or more of the members of the group or class are equally suitable or preferred; description of constituents in chemical terms refers to the constituents at the time of addition to any composition specified in the description, and does not necessarily preclude chemical interactions among the constituents of a mixture once mixed; the first definition of an acronym or other abbreviation applies to all subsequent uses herein of the same abbreviation and applies, mutatis mutandis, to normal grammatical variations of the initially defined abbreviation; and, unless expressly stated to the contrary, measurement of a property is determined by the same technique as previously or later referenced for the same property.

It is also to be understood that this invention is not limited to the specific embodiments and methods described herein, as specific components and/or conditions may, of course, vary. Furthermore, the terminology used herein is used only for the purpose of describing particular embodiments of the present invention and is not intended to be limiting in any way.

It must also be noted that, as used in the specification and the appended claims, the singular form “a,” “an,” and “the” comprise plural referents unless the context clearly indicates otherwise. For example, reference to a component in the singular is intended to comprise a plurality of components.

It will be understood that within this disclosure, any reference to a weight, weight ratio, and the like pertains to the dry matter, in particular to the dry matter of the composition.

Unless defined otherwise, all technical and scientific terms used herein generally have the same meaning as commonly understood by one of ordinary skill in the art to which this invention belongs.

As used herein, the term “comprising”, which is synonymous with “including” or “containing”, is open-ended, and does not exclude additional, unrecited element(s), ingredient(s) or method step(s), whereas the term “consisting of” is a closed term, which excludes any additional element, step, or ingredient which is not explicitly recited.

As used herein, the term “essentially consisting of” is a partially open term, which does not exclude additional, unrecited element(s), step(s), or ingredient(s), as long as these additional element(s), step(s) or ingredient(s) do not materially affect the basic and novel properties of the invention.

As used herein, the term “comprising” or “comprise(s)” hence includes the term “consisting of” or “consist(s) of”, as well as the term “essentially consisting of” or “essentially consist(s) of”. Accordingly, the term “comprising” or “comprise(s)” is, in the present application, meant as more particularly encompassing the term “consisting of” or “consist(s) of”, and the term “essentially consisting of” “essentially consist(s) of”.

Throughout this application, where publications are referenced, the disclosures of these publications in their entireties are hereby incorporated by reference into this application to more fully describe the state of the art to which this invention pertains.

The invention is hereinafter illustrated with reference to the following, non-limiting, example.

### EXAMPLE

### In Vitro Fermentation Model, TIM-2

The in vitro fermentation studies were done using the TIM-2 model. This is a validated, dynamic, computer-controlled model that simulates the human colon, mimicking body temperature, lumen pH, absorption of water and microbial metabolites through a semipermeable membrane inside the model, mixing and transporting the intestinal contents with peristaltic movements, using an anaerobic microbiota from human origin, it corresponds basically to the model as described in Minekus, M., et al. Appl. Microbiol. Biotechnol. 1999 53, 108 --- 114. doi: 10.1007/ s002530051622 and Kortman et al., Frontiers in Microbiology 2016, 6, 1481.

Characteristics of the movements of the contents in the TIM-2 system were simulated using an increase of the pH and peristaltic movements of the contents in the system using peristaltic pumps as described in Minekus, M (1998. Development and validation of a dynamic model of the gastrointestinal tract. PhD thesis, Delft University of Technology, The Netherlands).

### SCFA Analysis

SCFA analysis was performed at Brightlabs B.V., Venlo, The Netherlands), according to (Sáyago-Ayerdi SG, et al. Food Research International, E-pub date 13 Dec. 2017; Sayago Ayerdi et al Food Research International 118 (2019) 89-95).

### Inoculum

Pooled fecal microbiota samples from 11 healthy lean subjects not suffering from any metabolic disease was used to inoculate the TIM-2 in vitro fermentation model.

### Vitamine Mixture

A vitamin mixture was used containing (per liter): 1 mg menadione, 2 mg D-biotin, 0.5 mg vitamin B12, 10 mg pantothenate, 5 mg nicotinamide, 5 mg p-aminobenzoic acid and 4 mg thiamine.

### Dialysate

The dialysate used in the TIM-2 system contained (per liter): 2.5 g K₂HPO₄ ·3H₂O, 4.5 g NaCl, 0.005 g FeSO₄ ·7H₂O, 0.5 g MgSO₄ ·7H₂O, 0.45 g CaCl₂ ·2H₂O, 0.05 g bile and 0.4 g cysteine·HCl, plus 1 mL of the vitamin mixture.

### Example 1

The fecal microbiota was freshly sampled in and stored directly (within 2 h) on ice and under anaerobic conditions. Next, in an anaerobic cabinet, samples were diluted 1:1 with dialysate, and pooled at approximately equal weight, after which glycerol was added (to a final concentration of 12-13 w/w) and aliquots (30 ml/tube) were frozen in liquid nitrogen and stored at -80 ℃.

Prior to inoculation, 4x 30-ml aliquots were taken from the -80 ℃ freezer and thawed in a water bath at 37℃ for exactly 1 hour (still under anaerobic conditions). In an anaerobic cabinet, the microbiota from the 4 tubes was combined and the same volume of pre-reduced (i.e. oxygen-free) dialysate was added, gently mixed and divided over 4 syringes each comprising ca. 60 ml of microbiota-containing liquid. The syringes were sealed with a small flexible tube closed with a tubing clamp. Each TIM-2 unit was inoculated with 1 of the 4 syringes (i.e. 60 ml microbiota/dialysate mixture), using one single sample port to inoculate a TIM-2 unit. After the microbiota was introduced into the unit, another 60 ml of pre-reduced dialysate was added into the TIM-2 unit to get to a final volume of 120 ml per unit (i.e. system).

To simulate the conditions in the proximal region of the colon, the colon transversum and the distal part of colon, the pH of the microbiota/dialysate mixture was increased from pH 5.8 to pH 7.0 using 1 M NaOH over a period of 24 hours. The increase in pH simulated the traffic of fibers through the colon during the 24 hours’ experiment (wherein the last 16 hours simulated the more distal colonic site (i.e. transverse + distal)).

Samples (1 mL) were taken for SCFA analysis after 1, 2, 4, 6, 8 and 24 h after test product insertion; cumulative absolute amounts of SCFA were determined. Samples were centrifuged at 14,000 rpm for 10 min, filtered through a 0.45 µm PFTE filter, and diluted in the mobile phase (1.5 mM aqueous sulfuric acid). Ten microliters were loaded into the column with the help of an automatic sampler 730 (Metrohm, Herisa, Switzerland). The acids were eluted according to their pKa. The analysis was carried out by ion exclusion chromatography (IEC) using an 883 chromatograph (IC, Metrohm) equipped with a Transgenomic IC Sep ICE-ION-300 column (30 cm×7.8 mm×7 µm) and a MetroSep RP2 Guard, A column flow of 0.4 mL/min with a column temperature of 65° C. was used. The acids were detected using suppressed conductivity detection. Analyses were performed by Brightlabs (Venlo, The Netherlands).

### Addition of the Test-Product

After an adaption period of 40h, 7.5 grams of 2′-fucosyllactose (2′-FL), or 7.5 grams of 2′-FL and 7.5 grams of resistant starch (RS2 tapioca starch, Avebe,

Veendam, The Netherlands) were introduced into a TIM-2 unit through the sample port (Wednesday) as a single shot.

An experimental week contained the following steps:

Monday: Start up all 4 units of the TIM-2 system (pH 5.8).

Tuesday: Feeding of Simulated ileal efflux medium (SIEM) (Maathuis et al 2009 Journal of the American College of Nutrition 28(6):657-66 DOI: 10.1080/ 07315724.2009.10719798);

Simulated ileal efflux medium (SIEM) contained 5.7 g/liter BD Bacto tryptone (BD), 2.4 g/liter D-glucose (Sigma-Aldrich), 6.14 g/liter NaCl (Roth, Germany), 0.68 g/liter KH₂PO₄ (Merck, Germany), 0.3 g/liter NaH₂PO₄ (Merck, Germany), 1.01 g/liter NaHCO₃ (Merck, Germany), 5.6 g/liter bile salts no. 3 (Difco), 0.2 g/liter lysozyme (Serva, Germany), 1,000 U α-amylase (Fluka, Germany), 110 U trypsin (Sigma-Aldrich), 380 U chymotrypsin (Calbiochem, Germany), and 960 U lipase (Sigma-Aldrich). D(+)-Glucose and enzymes were filter sterilized before addition.

Wednesday: 3 h starvation period followed by a single insertion of test product; [2′-FL (7.5 gram of 2′-FL or 7.5 gram of 2′-FL + 7.5 gram of resistant starch) were added through the sample port]; following introduction of the test product, samples for SCFA analysis were taken after 1, 2, 4, 6 and 8 h.

Thursday: 24 h after insertion of the test product: last sample was taken for SCFA analysis;

Friday: cleaning, (the experiment was executed in one week).

### Results

The increase in acetate concentration between the last two sampling points (i.e. between 8 and 24 hours after insertion of sample) was taken as an indication for the increase in SCFA and acetate in the distal colon.

The results of the experiments are displayed below in Table 2.

TABLE 2

Results of the TIM-2 experiments, using microbiota samples from lean subjects to which either only 2′-FL was administered, or a combination of 2′-FL and resistant starch (RS).

| Test product | Lean (18.5 kg/m² ≤ BMI < 25 kg/m²) | |
|---|---|---|
| | Acetate | SCFA |
| 2′-FL w/o RS | 12 mmol | 21 mmol |
| 2′-FL with RS | 20.5 mmol | 30 mmol |

In Table 2, the amounts of acetate and short chain fatty acids (SCFA) refer to the amounts produced in between 8 and 24 hours after insertion of the test product; representing the amounts of acetate and SCFA produced in the distal colon.

The experiment shows that in lean subjects (having a BMI in a range of from at least 18.5 kg/m² to less than 25 kg/m²) SCFA levels and in particular acetate levels are increased in the distal colon when 2′-FL was added. When both 2′-FL and resistant starch were added, the amounts of SCFA and in particular acetate were even higher.

1. A non-therapeutic method for maintaining a healthy body weight or losing body weight, in a subject, the method comprising the step of administering 2′-fucosyllactose (2′-FL) and resistant starch to the subject,
wherein the subject is a mammal with a lean weightweight, preferably wherein the subject is a human, and
wherein the method does not comprise the step of administering mother’s milk to the subj ect.

2. A non-therapeutic method for (i) increasing the concentration of short chain fatty acid (SCFA), preferably of acetate, in the distal colon of a subject, (ii) for prevention of diet-induced body weight gain or adiposity, or (iii) for the improvement of glucose homeostasis and/or insulin sensitivity, wherein the non-therapeutic method comprises the step of administering 2′-fucosyllactose (2′-FL) to the subject, wherein said subject is as defined in claim 1 and wherein the method does not comprise the step of administering mother’s milk to the subject.

3. The non-therapeutic method according to claim 1, wherein the concentration of SCFA, preferably of acetate, in the distal colon of the subject is increased.

4. The non-therapeutic method according to claim 1, wherein the subject is a human aged 2 or more, with a body mass index (BMI) equal to or less than the upper value indicated in Table 1 for a healthy body weight for the age and sex of the subject:
TABLE 1
definition of healthy body weight BMI values

| Age | Male BMI (healthy body weight) | Female BMI (healthy body weight) |
|---|---|---|
| 2 | 15.14 - 18.40 | 14.83 - 18.01 |
| 3 | 14.74 - 17.88 | 14.47 - 17.55 |
| 4 | 14.43 - 17.54 | 14.19 - 17.27 |
| 5 | 14.21 - 17.41 | 13.94 - 17.14 |
| 6 | 14.07 - 17.54 | 13.82 - 17.33 |
| 7 | 14.04 - 17.91 | 13.86 - 17.74 |
| 8 | 14.15 - 18.43 | 14.02 - 18.34 |
| 9 | 14.44 - 19.09 | 14.28 - 19.06 |
| 10 | 14.64 - 19.83 | 14.61 - 19.85 |
| 11 | 14.97 - 20.54 | 15.05 - 20.73 |
| 12 | 15.35 - 21.21 | 15.62 - 21.67 |
| 13 | 15.84 - 21.90 | 16.26 - 22.57 |
| 14 | 16.41 - 22.61 | 16.88 - 23.33 |
| 15 | 16.98 - 23.28 | 17.45 - 23.93 |
| 16 | 17.54 - 23.89 | 17.91 - 24.36 |
| 17 | 18.05 - 24.45 | 18.25 - 24.69 |
| ≥18 | 18.50 - 24.99 | 18.50 - 24.99 |

.

5. The non-therapeutic method according to claim 1, wherein the administering is an oral administration.

6. The non-therapeutic method according to claim 1, wherein no LNnT is administered to the subject.

7. The non-therapeutic method according to claim 1, wherein the 2′-fucosyllactose is comprised in a composition optionally comprising resistant starch.

8. The non-therapeutic method according to claim 7, wherein the composition comprises an amount of 2′-fucosyllactose of at least 0.5 g and optionally comprises an amount of resistant starch of at least 0.5 g.

9. The non-therapeutic method according to claim 6, wherein the weight ratio of 2′-fucosyllactose to resistant starch is in the range of from 0.5:10 to 10:0.5.

10. The non-therapeutic method according to claim 6, wherein the combined amount of 2′-FL and resistant starch is at most 30 gram, preferably at most 25 gram, more preferably at most 20 gram, most preferably at most 15 gram.

11. The non-therapeutic method according to claim 6, wherein composition is a food product.

12. The non-therapeutic method according to claim 6, wherein the composition is in the form of single servings.

13. A method for the prevention of overweight or of a condition associated with overweight in a subject, the method comprising administering to the subject a composition comprising (i) 2′-fucosyllactose, and (ii) resistant starch , wherein the subject is as defined in claim 1.

14. A method for maintaining a healthy body weight or losing body weight in a subject, the method comprising administering to the subject a composition comprising 2′-fucosyllactose, and resistant starch, wherein said subject is as defined in claim 1.

15. A composition comprising at least 5 wt% of 2′-fucosyllactose (2′-FL) and at least 5 wt% of resistant starch as determined on the dry matter.

16. The non-therapeutic method according to claim 11, wherein the food product is selected from the group consisting of dairy product, bar, liquid product, dessert-type product, savory snack, savory biscuit, bakery product, pasta, and food supplement.

17. The non-therapeutic method according to claim 11, wherein the food product is in the form of a single serving, and optionally such single serving is individually packaged.
